(19) United States
(12) Patent Application Publication
(10) Pub. No.: US 2002/0123655 A1
(43) Pub. Date: Sep. 5, 2002

(54) Process for preparing alkoxy- and aryloxy-phenols

(75) Inventors: Lung-Huang Kuo, Kaohsiung City (TW); Chih-Da Lin, Taichung City (TW); Shyh-Shyan Jwo, Tien-Chung Chen (TW); Li-Ling Lin, Kaohsiung City (TW); Shou-Lun Lee, Tai-Ping City (TW); Shi-Jen Tsai, Changhua Hsien (TW)

Correspondence Address: LADAS & PARRY, 26 West 61st Street, New York, NY, 10023-7604, US

(73) Assignee: SINON CORPORATION

(21) Appl. No.: 09/788,717

(22) Filed: Feb. 20, 2001

(51) Int. Cl. C07C 41/01

(52) U.S. Cl. 568/650

(57) ABSTRACT

A process for preparing alkoxy- and aryloxy-phenols involves the steps of reacting a benzaldehyde derivative with a peracid to form a benzodioxole derivative, removing non-reacted peracid from the reaction mixture, and reacting the benzodioxole derivative in the reaction mixture with a hydrolyzing agent.

### BACKGROUND OF THE INVENTION

1. Field of the Invention

This invention relates to a process for preparing alkoxy- and aryloxy-phenols by oxidizing an alkoxy- or aryloxy-benzaldehyde with a peracid to form an alkoxy- or aryloxy-benzodioxole derivative and subsequently hydrolyzing the alkoxy- or aryloxy-benzodioxole derivative with a hydrolyzing agent in the presence of an acid.

2. Description of the Related Art

U.S. Pat. No. 5,840,997 disclosed a method for conversion of an alkoxy- or aryloxy-benzaldehyde having the following formula

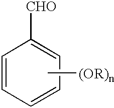

to an alkoxy- or aryloxy-phenol having the following formula,

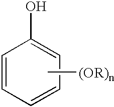

wherein R can be an alkyl or an aryl group and n is an integer number ranging from 1 to 3. The method involves the steps of converting an aldehyde group on the alkoxy- or aryloxy-benzaldehyde to a formate ester group (—OCHO), and subsequently converting the formate ester group to a hydroxy group. The conversion of the aforesaid aldehyde group to the formate ester group is carried out by oxidizing the former with a peracid, such as performic acid, and the conversion of the aforesaid formate ester group to the hydroxy group is carried out by saponification of the former with a caustic solution, such as aqueous sodium hydroxide. The performic acid is formed in-situ by the reaction of hydrogen peroxide with formic acid in the reaction mixture during the aforesaid oxidation stage.

The aforesaid method is disadvantageous in that since the concentration of the alkoxy- or aryloxy-benzaldehyde in the reaction mixture during the aforesaid oxidation stage has to be kept at a lower level, the reaction rate of the oxidation is retarded. Moreover, a large amount of the caustic solution is required to neutralize the remaining performic acid, formic acid, and hydrogen peroxide, which results in formation of enormous amounts of sludge waste, and the need for repeated acid and base washings and extractions during purification of the product.

### SUMMARY OF THE INVENTION

Therefore, it is an object of the present invention to provide a process for preparing alkoxy- and aryloxy-phenols that is capable of overcoming the aforesaid drawbacks.

According to the present invention, there is provided a process for preparing alkoxy- and aryloxy-phenols having a formula (I),

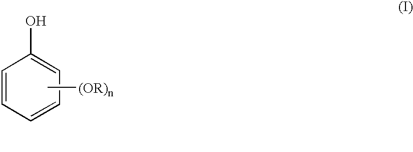

Wherein R is an alkyl or an aryl group and n is an integer number ranging from 1 to 3. The process comprises the steps of: reacting a benzaldehyde derivative having a formula (II)

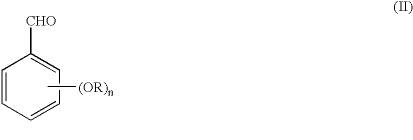

with a peracid selected from a group consisting of performic acid, peracetic acid, perbenzoic acid, and m-chloroperbenzoic acid to form a benzodioxole derivative having a formula (III);

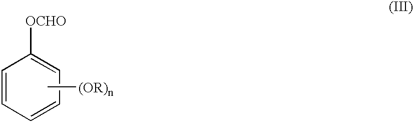

removing the non-reacted peracid from the reaction mixture; and reacting the benzodioxole derivative in the reaction mixture with a hydrolyzing agent selected from a group consisting of water, tetrahydrofuran, an alcohol selected from a group consisting of methanol, ethanol, 1-propanol, 2-propanol, 1-butanol, 2-butanol, 2-methyl-1-propanol, and 2-methyl-2-propanol, and mixtures thereof in the presence of an acid selected from a group consisting of formic acid, acetic acid, hydrochloric acid, sulfuric acid, phosphoric acid, and mixtures thereof.

### DETAILED DESCRIPTION OF THE PREFERRED EMBODIMENT

Preparation of alkoxy- and aryloxy-phenols (formula (I)) according to the process of this invention is described in detail as follows.

The process for preparing alkoxy- and aryloxy-phenols comprises the steps of: oxidizing the benzaldehyde derivative (formula (II)) by reacting with a peracid selected from a group consisting of performic acid, peracetic acid, perbenzoic acid, and m-chloroperbenzoic acid to form a benzodioxole derivative (formula (III)); removing the non-reacted peracid from the reaction mixture; and hydrolyzing the benzodioxole derivative in the reaction mixture by reacting with a hydrolyzing agent selected from a group consisting of water, tetrahydrofuran, an alcohol selected from a group consisting of methanol, ethanol, 1-propanol, 2-propanol, 1-butanol, 2-butanol, 2-methyl-1-propanol, and 2-methyl-2-propanol, and mixtures thereof in the presence of an acid selected from a group consisting of formic acid, acetic acid, hydrochloric acid, sulfuric acid, phosphoric acid, and mixtures thereof.

Preferably, the peracid is formed in situ in the reaction mixture during the oxidation stage by reaction of hydrogen peroxide with a corresponding acid, such as formic acid ,acetic acid, acetic anhydride, etc..

The following Examples illustrate preparation of Sesamol according to the process of this invention. Sesamol is an alkoxy-phenol having the following formula

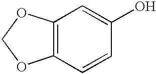

and is known as an intermediate for production of paroxetine, which is used as an anti-depressant drug.

Formation of 1,3-benzodioxole-formate ester

### EXAMPLE 1

710 ml of acetic anhydride was charged into a 3-necked 1 L round bottom flask equipped with a stirrer and a condenser. The acetic anhydride was heated to a temperature of about 40° C. Three portions of 50% hydrogen peroxide, each of which is in an amount of 36.3 g (0.53 mole), were separately added into the flask at an interval of every two hours. 80 g (0.53 mole) of Piperonal was charged into the flask, and the reaction mixture was stirred for 2 hours at a temperature of about 40° C. and was subsequently concentrated via a rotary evaporator to yield a brown liquid. The brown liquid was then subjected to vacuum distillation, which operated at a temperature of about 80-83° C. and a vacuum pressure of 3 mmHg, to yield a pale yellow liquid product in an amount of 78.8 g with a yield of 89%. The purity of the product was determined by liquid chromatography to be greater than 97% of 1,3-benzodioxole-formate ester.

### EXAMPLE 2

530 ml of acetic anhydride was charged into a 3-necked 1 L round bottom flask equipped with a stirrer and a condenser. The acetic anhydride was heated to a temperature of about 40° C. 81.6 g (1.2 mole) of 50% hydrogen peroxide was added into the flask. The mixture was stirred for 3 hours. 60 g (0.4 mole) of Piperonal was charged into the flask, and the reaction mixture was stirred for 2 hours at a temperature of about 40° C. and was subsequently concentrated via a rotary evaporator to yield a brown liquid. The brown liquid was then subjected to vacuum distillation, which operated at a temperature of about 80-83° C. and a vacuumpressure of 3 mmHg, to yield a pale yellow liquid product in an amount of 57.8 g with a yield of 87%. The purity of the product was determined by liquid chromatography to be greater than 97% of 1,3-benzodioxole-formate ester.

Formation of Sesamol

### EXAMPLE 3

52.8 g (0.32 mole) of 1,3-benzodioxole-formate ester, 100 ml of methanol, and 2 ml of acetic acid were charged into a 250 ml round bottom flask with a condenser. The mixture was heated to reflux for 12 hours. Afterwards, the reaction mixture was concentrated via a rotary evaporator to yield a pale brown liquid, and was subsequently subjected to vacuum distillation at a temperature of about 101-104° C. and a vacuum pressure of 3 mmHg to yield a pale yellow liquid product in an amount of 41.3 g with a yield of 94%. The purity of the product was determined by liquid chromatography to be greater than 99%.

### EXAMPLE 4

57.8 g (0.35 mole) of 1,3-benzodioxole-formate ester, 100 ml of methanol, and 10 ml of acetic acid were charged into a 250 ml round bottom flask with a condenser. The mixture was heated to reflux for 5 hours. Afterwards, the reaction mixture was concentrated via a rotary evaporator to yield a pale brown liquid, and was subsequently subjected to vacuum distillation at a temperature of about 101-104° C. and a vacuum pressure of 3 mmHg to yield a pale yellow liquid product in an amount of 39.9 g with a yield of 83%. The purity of the product was determined by liquid chromatography to be greater than 99%.

### EXAMPLE 5

15.8 g (95.2 mmol) of 1,3-benzodioxole-formate ester, 100 ml of methanol, and 0.2 ml of phosphoric acid were charged into a 250 ml round bottom flask with a condenser. The mixture was heated to reflux for 5 hours. Afterwards, the reaction mixture was concentrated via a rotary evaporator to yield a pale brown liquid, and was subsequently subjected to vacuum distillation at a temperature of about 101-104° C. and a vacuum pressure of 3 mmHg to yield a pale yellow liquid product in an amount of 10.1 g with a yield of 77%. The purity of the product was determined by liquid chromatography to be greater than 99%.

### EXAMPLE 6

26.2 g (0.16 mole) of 1,3-benzodioxole-formate ester, 100 ml of methanol, and 0.5 ml of 37% hydrochloric acid were charged into a 250 ml round bottom flask with a condenser. The mixture was heated to reflux for 3 hours. Afterwards, the reaction mixture was concentrated via a rotary evaporator to yield a pale brown liquid, and was subsequently subjected to vacuum distillation at a temperature of about 101-104° C. and a vacuum pressure of 3 mmHg to yield a pale yellow liquid product in an amount of 19.6 g with a yield of 90%. The purity of the product was determined by liquid chromatography to be greater than 99%.

### EXAMPLE 7

26.2 g (0.16 mole) of 1,3-benzodioxole-formate ester, 100 ml of 2-propanol, and 5 ml of acetic acid were charged into a 250 ml round bottom flask with a condenser. The mixture was heated to reflux for 14 hours. Afterwards, the reaction mixture was concentrated via a rotary evaporator to yield a pale brown liquid, and was subsequently subjected to vacuum distillation at a temperature of about 101-104° C. and a vacuum pressure of 3 mmHg to yield a pale yellow liquid product in an amount of 17.9 g with a yield of 82%. The purity of the product was determined by liquid chromatography to be greater than 99%.

### EXAMPLE 8

15.0 g (90.4 mmol) of 1,3-benzodioxole-formate ester, 50 ml of methanol, 50 ml of water, and 5 ml of acetic acid were charged into a 250 ml round bottom flask with a condenser. The mixture was heated to reflux for 3 hours. Afterwards, the reaction mixture was concentrated via a rotary evaporator to yield a pale brown liquid, and was subsequently subjected to vacuum distillation at a temperature of about 101-104° C. and a vacuum pressure of 3 mmHg to yield a pale yellow liquid product in an amount of 11.5 g with a yield of 92%. The purity of the product was determined by liquid chromatography to be greater than 99%.

### EXAMPLE 9

15.0 g (90.4 mmol) of 1,3-benzodioxole-formate ester, 50 ml of methanol, 50 ml of water, and 5 ml of tetrahydrofuran were charged into a 250 ml round bottom flask with a condenser. The mixture was heated to reflux for 8 hours. Afterwards, the reaction mixture was concentrated via a rotary evaporator to yield a pale brown liquid, and was subsequently subjected to vacuum distillation at a temperature of about 101-104° C. and a vacuum pressure of 3 mmHg to yield a pale yellow liquid product in an amount of 11.3 g with a yield of 91%. The purity of the product was determined by liquid chromatography to be greater than 99%.

### EXAMPLE 10

440 ml of acetic anhydride was charged into a 3-necked 1 L round bottom flask equipped with a stirrer and a condenser. The acetic anhydride was heated to a temperature of about 40° C. Three portions of 50% hydrogen peroxide, each of which is in an amount of 22.7 g (0.33 mole), were separately added into the flask at an interval of every two hours. 50 g (0.33 mole) of Piperonal was charged into the flask, and the reaction mixture was stirred for 2 hours at a temperature of about 40° C. to oxidize Piperonal and was subsequently concentrated via a rotary evaporator to yield a brown liquid. All of the brown liquid and 500 ml of methanol and 10 ml of acetic acid were charged into a flask with a condenser. The mixture was heated to reflux for 12 hours. Afterwards, the reaction mixture was concentrated via a rotary evaporator to yield a pale brown liquid, and was subsequently subjected to vacuum distillation at a temperature of about 101-104° C. and a vacuum pressure of 3 mmHg to yield a pale yellow liquid product in an amount of 37.3 g with a yield of 81%. The purity of the product was determined by liquid chromatography to be greater than 98%.

It is noted that without concentration of the reaction mixture to remove most of the remaining acetic anhydride and the non-reacted peracetic acid after the oxidation stage, subsequent hydrolyzation of the intermediate (i.e. the benzodioxole derivative) in the reaction mixture will cause decomposition of the benzodioxole derivative.

With the invention thus explained, it is apparent that various modifications and variations can be made without departing from the spirit of the present invention. It is therefore intended that the invention be limited only as recited in the appended claims.

We claim:

1. A process for preparing alkoxy- and aryloxy-phenols having a formula (I), 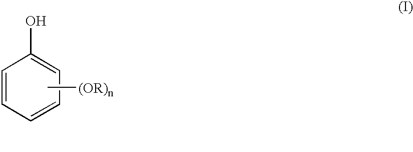
wherein R is an alkyl or an aryl group and n is an integer number ranging from 1 to 3, the process comprising the steps of:
reacting a benzaldehyde derivative having a formula (II) 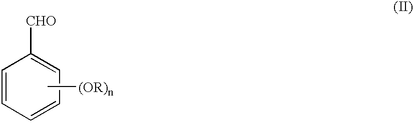
with a peracid selected from a group consisting of performic acid, peracetic acid, perbenzoic acid, and m-chloroperbenzoic acid to form a benzodioxole derivative having a formula (III); 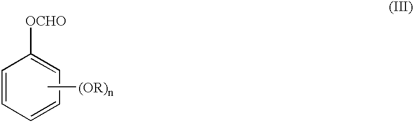
removing the non-reacted peracid from the reacti o n mixture; and
reacting the benzodioxole derivative in the reaction mixture with a hydrolyzing agent selected from a group consisting of water, tetrahydrofuran, an alcohol selected from a group consisting of methanol, ethanol, 1-propanol, 2-propanol, 1-butanol, 2-butanol, 2-methyl-1-propanol, and 2-methyl-2-propanol, and mixtures thereof in the presence of an acid selected from a group consisting of formic acid, acetic acid, hydrochloric acid, sulfuric acid, phosphoric acid, and mixtures thereof.

2. The process of claim 1, wherein the hydrolyzing agent is methanol, and the acid is acetic acid.

3. A process for preparing alkoxy- and aryloxy-phenols having a formula (I), 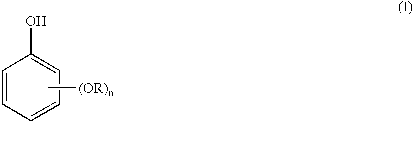
wherein R is an alkyl or an aryl group and n is an integer number ranging from 1 to 3, the process comprising the step of:
reacting a benzodioxole derivative having a formula (III); 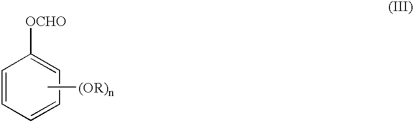
with a hydrolyzing agent selected from a group consisting of water, tetrahydrofuran, an alcohol selected from a group consisting of methanol, ethanol, 1-propanol, 2-propanol, 1-butanol, 2-butanol, 2-methyl-1-propanol, and 2-methyl-2-propanol, and mixtures thereof in the presence of an acid selected from a group consisting of formic acid, acetic acid, hydrochloric acid, sulfuric acid, phosphoric acid, and mixtures thereof.

4. The process of claim 1, wherein the hydrolyzing agent is methanol, and the acid is acetic acid.
